(19) United States
(12) Patent Application Publication
(10) Pub. No.: US 2011/0306034 A1
(43) Pub. Date: Dec. 15, 2011

(54) METHOD FOR ISOLATING CELLS AND DISEASE VECTORS FROM BODILY FLUIDS

(76) Inventor: Hans-Werner Heinrich, Greifswald (DE)

(21) Appl. No.: 12/995,256

(22) PCT Filed: May 26, 2009
(86) PCT No.: PCT/DE2009/000719

§ 371 Date: Jan. 7, 2011

(30) Foreign Application Priority Data
May 30, 2008 (DE) 102008025965.9

(51) Int. Cl. C12Q 1/70; C12Q 1/02; C12N 13/00; C12M 1/00; C12N 11/00; C12N 11/08

(52) U.S. Cl. 435/5; 435/174; 435/180; 435/173.9; 435/283.1; 435/29

(57) ABSTRACT

Embodiments provide one of a method, a device, and their use to isolate or analyze cells, including pathogens from body fluids by means of different separation methods.

The task is solved by a method for the isolation of somatic cells, micro organisms and/or virus from body fluid from individuals, comprising the steps of
a) Isolation of immune complexes from body fluids of an individual;
b) Cleavage of the isolated immune complexes in their sub units;
c) Binding of the dissociated sub units on solid support;
d) Incubation of the modified solid support from (c) with cells from body fluids of the individual (a); and
e) Isolation of the incubated solid support from (d) with the cells, micro organisms and/or virus bound to the sub units;
   as well as a device especially suitable for diagnostic and therapy, comprising
A solid support, preferably micro particles
Sub units from immune complexes bound to the solid support, preferably from CIC of an individual
Cells, micro organisms, and/or virus, preferably MNCs from the same individual and their usage.

### CROSS-REFERENCE TO RELATED APPLICATIONS

This application is the United States national phase under 35 U.S.C. §371 of PCT International Application No. PCT/DE2009/000719, filed on May 26, 2009, and claiming priority to German application no. 10 2008 025 965.9, filed on May 30, 2008. Both priority applications are incorporated by reference herein.

### BACKGROUND OF THE INVENTION

1. Field of the Invention

The invention refers to the production of separation systems for cells and/or pathogens based on sub units of circulating immune complexes (CIC).

2. Background of the Art

The functional basis of the immune reaction is based on the fine tuned collaboration of local and systemic acting cellular and humeral components of innate and acquired immunity. CIC are the product of former action and reaction between these components, e.g. antibodies and antigens, receptors and the matching ligands, complement factors, albumin, and other plasma factors. As a result, distinct agglomerates were formed which are incorporated and disintegrated by phagocytes. They are jointly responsible for the beginning and continuation of a number of acute and chronic inflammatory diseases within the immune pathological events.

CIC are the morphologic end product, and in that way the reflection of ongoing or previous processes of immune regulation. This is true for the physiological life maintaining immune response, as well as for immune pathologic processes which can lead to disease and death. CIC can be found in the blood of every individual. Increased levels are seen in connection of e.g. rheumatoid arthritis.

The analysis of the composition of the CIC by cleavage and analysis of the sub units would be a snap shot of the immune regulatory proceedings in an individual.

The sub units can be gently separated. They keep their function to bind the appropriate reaction partner. The sub units can be coupled to a solid support. This procedure is described in DE 19538641 Like an affinity chromatography, it allows the removal of CIC and their sub units from the blood of those individuals from whom the CIC have been isolated. The impact of plasmapheresis on the immune regulation could be proven the by a small animal experiment with rats of the inbreed lineage BB/OK. BB/OK rats develop an auto-aggressive β-cell destruction similar to that of the human juvenile diabetes (diabetes type 1). The CICs separated from plasma collections were dissociated and covalently coupled to Sepharose. It was possible to stop the process of β-cell self destruction in pre-diabetic rats with proven islet cell inflammation by extra-corporeal, temporal treatment (Berg, S. et al. Diab. Stoffwechsel, 11 (2002), Suppl. 1 P. 55).

### Pathogenic Basis, Auto Immunity, Allergies and Tumors

A number of immune competent cells and signal systems contribute to the efficient defense of infectious agents and toxic substances as well as in the homeostasis of healthy somatic cells. Antibodies receptors and soluble mediators play an essential function in this complex process. Antibodies are specifically directed to self and no-self antigens and participate by different mechanisms (e.g. complement activation) in their elimination. Balance and correct function depend on a number of factors.

Auto immunity is characterized by the loss of tolerance toward the body's own tissue. Multiple exogenous and endogenous factors participate in the deregulation of the immune system. Many mechanisms are discussed which may be involved in the pathogenesis of auto immune diseases. Cross reacting antibodies or an antigen driven specific immune response are two out of many mechanisms. The inadequate control of potential auto-reactive cells and the presentation of auto antigens lead to the formation of auto reactive cells and the production of pathogenic antibodies with the result of an extensive destruction, and not infrequently with life threatening consequences.

Antibody connected disorders form a wide spectrum, reaching from functional disturbance of one receptor up to systemic, self destructive disease.

The clinical picture depends on the specificity of the auto immune reaction. Organ specific diseases like Morbus Basedow (Graves' disease) represent one side of the spectrum. On the other side, systemic auto immune diseases are assigned, in which the rheumatic diseases are also counted (e.g. rheumatoid arthritis). Here, lesions and antibodies are not restricted to one organ. In addition, mixed forms and intermediates are described, like e.g. Myasthenia gravis.

As a consequence, it is necessary to remove the pathogenic important antibodies, respectively the CICs which contain the auto antibodies to influence the pathologic process positively, and control the associated symptoms.

Allergies also become manifested based on a malfunction of the immune system.

The primary contact with certain antigens (allergens) induces in genetically predisposed people a malfunction in the balance between Ig-subclasses, IgE-receptor distribution, T1:TH2-relation (with it the cytokine synthesis), and IgE-synthesis. The results of this malregulation are the known acute, respectively chronic symptoms after repeated allergen contact.

The result of the dysregulation is also, aside from the increased IgE and IgE-receptor synthesis, a significant elevation of IgG-anti-IgE-auto-antibodies which can be detected in circulating immune complexes, and correlates with the serum IgE level (e.g. up to 32% of the total serum IgE in patients with atopic dermatitis could be detected in form of IgG-anti-IgE-immune complexes). But, a correlation between the concentration of IgG-anti-IgE-immune complexes and the severity of the disease does not exist, based on the known heterogeneity of the antibodies in their capacity to induce the histamine release in basophiles.

Unquestionably, auto antibodies play an immune regulatory role which is not yet fully known. Moreover, IgG-anti-IgE can have an IgE-allergen-complex eliminating function due to the high affinity of C1q to IgG1 and the Fc gamma-receptor.

The previous origin of a (more or less) causal therapy consists of the inactivation of free IgE with the exception of the treatment with Nedocromil, which probably prevents the sub class switch to IgE during the B-cell maturation. Besides the application of high doses of donor IgG, which generate convincing resultants in individual cases, the use of a recombinant humanized monoclonal antibody (rhuMAb-E25, a joint development of the companies Genentech, Norvatis Pharma, and Tanox Biosystems) has gone the furthest on the way until the Approval. This antibody binds to the Fc-receptor binding region of free IgE and prevents its docking to the different IgE-receptors. That makes it possible to reduce the concentration of free IgE to more than 95%. The elimination of the mAb-IgE-complex takes place by the described way of IgR-FcR-binding.

The disadvantage of the application of this antibody will be the cost factor, which will be significant for patients who have a very high IgE concentration.

Besides the inactivation of free IgE's by antibodies, the extra-corporeal elimination could be a further promising therapeutic method, at least for certain patients.

Experiments to this were accomplished in the former Soviet Union and in Japan by using immune adsorbers, carrying covalently linked specific anti-IgE antibodies as ligand. Side effects were not noticed. The concentration of free IgE was reduced to about 83-98%, and a therapeutic effect could be demonstrated during a monitoring time of 6 months. More differentiated treatment protocols are, unfortunately, not available.

Tumors escape the immunologic defense by the liberation of different factors which induce immune tolerance. As a consequence, the unlimitedly growing tumor is seen as an “embryo” by the defense system. Specific antibodies are formed directed to tumor proteins, the so called neo-antigens, or specific binding ligands are present. Antibodies, as well as ligands, result in a complex formation with the neo-antigens. The immunologic reaction is suppressed by the following mechanisms, e.g.:
Liberation of immune suppressive factors (like soluble tumor necrosis factor receptor (sTNFRs) and neo-antigens like CEA, MUC1, CA15-3, etc.). The blood concentration of neo-antigen immune complexes has prognostic relevance for certain tumors
Interference with regulative T-cells (TGFβ-increase leads to the decrease of cytotoxic T-cells)
Enlarged expression of ILT3 and ILT4 on dendritic cells induces immune tolerance
Antigen modulation
Cytophilic antibodies mask tumor antigens

Additional growth advantage is generated for the growing tumor by the loss of tissue integration as a result of an increased expression of NFAT_{tumor cells} (nuclear factor of activated T-cells) and α-6-β-4-integrin; the suppression of apoptosis (TOR-Sir3-hsp, reaper-DIAP, Dbc2 protein apoptosis) and/or increased cell division (HER-network—EGFR; ras—IL24/IL24receptor); and vascularisation of solid tumors (CUGBP2-COX2-prostaglandins).

The condensation products of tumor action and the body counteraction circulate as CIC in the blood.

Plasma exchange and protein A immune adsorption have been tested for tumor treatment for more than 20 years. A temporal positive effect could be demonstrated in certain cases.

### Virus Infections

The interaction of virus and antibodies normally results in the neutralization of viruses. These virus immune complexes are incorporated and disintegrated by phagocytes. It is known from some viruses, like Dengue virus, that they show an even higher infectivity bound to immune complexes in comparison to the free virus. Neutralized Herpes virus IgG immune complexes induce the same amount of IL6 release in macrophages like free virus DNA. A correlation between distemper virus immune complexes and rheumatoid arthritis could be proven in dogs (May et al. Rheumatology, 33 (1994), 27-31). Virus-immune complexes play a pathogenic role in virus induced immune complex diseases (e.g. glomerulo-nephritis, Appel et al. NEJM, 328 (1993), 506-509). Nearly all patients suffering from a chronic Hepatitis C infection are carriers of immune complex bound HCV (Morita et al. Hepato-Gastroenterology, 43 (1996), 582-585), which is responsible for the continuous re-infection. Any therapeutic interaction of the mostly chronic virus infection with extra-corporeal blood treatment procedures must also include the removal of immune complex bound virus.

### BRIEF SUMMARY OF THE INVENTION

Whereas the analysis of individual factors and their role in the physiologic and pathologic immune reaction generated a fast gain of knowledge, thanks to the molecular research methods, the investigation of the regulatory interaction on a cellular level is still a methodic challenge.

The goal of the invention is to provide a procedure and a configuration and their usage, whereby on the basis of different separation procedures, cells, including pathogens from body liquids, can be isolated and analyzed. Therefore, for the first step it is necessary to detect the target cells.

The basis of the invention is the CICs. They are not only the end product of in a biologic chain of reaction, intended to get degraded. CIC are also the key for the isolation of cells from the blood of individuals, which are related to the actual status of the cellular immune reaction. Surprisingly, it could be demonstrated, that the sub-units of the CIC bind on the surface of cells.

### BRIEF DESCRIPTION OF THE FIGURES

FIG. 1.: Total fraction of dissociated CIC coupled on particles and incubated with mononuclear cells (MNC) of the donor J.M. after Ficoll gradient isolation.

FIG. 2.: Total fraction of dissociated CIC coupled on particles and incubated with whole blood of the donor J.M.

FIG. 3.: 300-100 kDa fraction of dissociated CIC coupled on particles and incubated with mononuclear cells (MNC) of the donor J.M. after Ficoll gradient isolation.

FIG. 4.: 300-100 kDa fraction of dissociated CIC coupled on particles and incubated with whole blood of the donor J.M.

### DETAILED DESCRIPTION OF THE INVENTION

CICs were isolated from individual plasma with common methods like precipitation or protein A adsorption. After such isolation, the CICs were split in their biologic active sub units, preferably done by lowering the pH to <3.0.

Now, the sub units can be separated by known methods, e.g. gel chromatography, and consecutively coupled separately and discrete, and individually, or as mixture on an appropriate solid support, preferably micro particles, wherein all available common solid support materials are usable, especially polystyrenes or especially preferred Sepharose. By means of these “capture particles”, cells can be separated from blood and other body liquids by the application of different separation procedures. In a second step, the cells can be subsequently characterized, in vitro cultivated, in sub populations divided, manipulated, and used for further diagnostic and therapeutic purposes.

Moreover other continuous or discontinuous methods of extra-corporeal cell depletion for therapeutic application can be carried out with the procedure according to the present invention. Those systems can be re-activated with known methods and can be used repeatedly.

Embodiments of the invention are described with the following examples:

### Example 1

Drawing of anti coagulated blood from the donor J.M. by use of a common blood drawing system; 4 ml whole blood centrifuged (10 min 600×g); aspirate of 2 ml and combine with 2 ml 0.1 M borate buffer; adding of 4 ml 7% PEG in borate buffer, vortex and keep refrigerated overnight for the precipitation reaction.

Precipitate centrifuged for 30 min (4° C., 1,600×g); remove and discard the supernatant; washing the sediment twice with 10 ml 3.5% PEG in borate buffer, 2×1 min vortex and centrifuge 30 min 1.600×g at 4° C.; remove and discard the supernatant.

The sediment (CIC) is re-suspended in 1 ml PBS (pH 7.4).

The pH of the CIC solution will be adjusted to 3.0 by HCl

### Protein Coupling:

Activation of the polystyrene particles with NHS and EDC in MES puffer; washing of the activated particles in MES pH 3.0 and re-suspension in MES pH 3.0; adding the protein solution and shaking for the time of protein binding on the polystyrene particles. Stopping of free binding positions by glycerin or ethanolamine; washing of the polystyrene particles with PBS and resuspend in PBS.

Incubation of Cells with the Particles:
a) Mononuclear cells (MNC) from the donor J.M. isolated by Ficoll-gradient centrifugation. 3 million MNC were suspended in 300 μl PBS/BSA puffer pH 7.4 and 20,000 CIC-coupled polystyrene are added; the reaction tube will be moved on a tilting-rolling mixer for 45 min at room temperature; add 3 ml PBS/BSA buffer to the suspension; isolation the particles by means of sieves; 3 times washing with 5 ml PBS/BSA buffer pH 7.4 and resuspend in 1 ml PBS/BSA buffer in a multiwell plate for further analysis.
b) Whole blood from the donor J.M.

Centrifuge 2 ml whole blood 10 min, 350×g; remove and discard the plasma; blood pellet re-suspended in 1 ml PBS/BSA buffer pH 7.4, vortex and centrifuge for 10 min 350×g; repeating of the washing two times; discarding of the supernatant and re-suspending the blood cells in 1 ml PBS/BSA; adding of 20,000 CIC-coupled particles and incubating the sample for 45 min at room temperature (RT) on a tilting-rolling-mixer; adding 3 ml PBS/BSA into the mixing container and isolating the particles by a sieve and washing with 10 ml PBS/BSA pH 7.4; re-suspended in 1 ml PBS/BSA buffer in a multiwell plate for further analysis.

Adding of Calcein AM and propidium iodine; analyzing by means of a fluorescence microscope.

### Example 2

Centrifugation of 4 ml whole blood for 10 min 600×g; transferring 2 ml plasma in a sample tube and adding of 2 ml 0.1 M borate buffer; adding of 4 ml 7% PEG in borate buffer, vortex and keep refrigerated overnight for the precipitation reaction.

Precipitate centrifuged for 30 min 1,600×g, 4° C.; removal and discarding the supernatant; washing of the sediment with 10 ml 3.5% PEG in borate buffer; 2×1 min vortex and centrifuged for 30 min 1,600×g, 4° C.;

Discarding of the supernatant and re-suspension of the sediment in 1 ml PBS pH 7.4 Adjustment of the pH to 3.0 by HCl.

Separation of a 30 kDa-100 kDa fraction.

Adding of 4 ml PBS pH 2.7 to the protein solution in a Amicon Ultra 100K device; centrifuged for 20 min 4,000×g, 4° C.; transfer of the flow-through in a Amicon Ultra 30K device and spin for 30 min at 4,000×g at 4° C.; repeating of the procedure twice; protein solution in Amicon Ultra 30K washing two times with 4 ml PBS pH 3.0; combining the 30 kDa-100 kDa fractions and estimation of the protein concentration.

### Protein Coupling:

Activation of the polystyrene particles with NHS and EDC in MES puffer; washing of the activated particles in MES pH 3.0 and re-suspension in MES pH 3.0; adding the protein solution and shaking for the time of protein binding on the polystyrene particles. Stopping of free binding positions by glycerin or ethanolamine; washing of the polystyrene particles with PBS and resuspend in PBS.

Incubation of the Particles with Cells:
a) Mononuclear cells (MNC) from the donor H.W. after Ficoll gradient isolation 3 million MNC were suspended in 300 μl PBS/BSA puffer pH 7.4 and 20,000 CIC-coupled polystyrene are added; the reaction tube will be moved on a tilting-rolling mixer for 45 min at room temperature; add 3 ml PBS/BSA buffer to the suspension; isolation the particles by means of sieves; 3 times washing with 5 ml PBS/BSA buffer pH 7.4 and resuspend in 1 ml PBS/BSA buffer in a multiwell plate for further analysis.
b) Whole blood from the donor H.W.

Centrifuge 2 ml whole blood 10 min, 350×g; remove and discard the plasma; blood pellet re-suspended in 1 ml PBS/BSA buffer pH 7.4, vortex and centrifuge for 10 min 350×g; repeating of the washing two times; discarding of the supernatant and re-suspending the blood cells in 1 ml PBS/BSA; adding of 20,000 CIC-coupled particles in the sample tube and incubating the sample for 45 min at room temperature (RT) on a tilting-rolling-mixer; adding 3 ml PBS/BSA into the mixing container and isolating the particles by a sieve and washing with 10 ml PBS/BSA pH 7.4; re-suspended in 1 ml PBS/BSA buffer in a multiwell plate for further analysis.

Adding of Calcein AM and propidium iodine; analyzing by means of a fluorescence microscope.

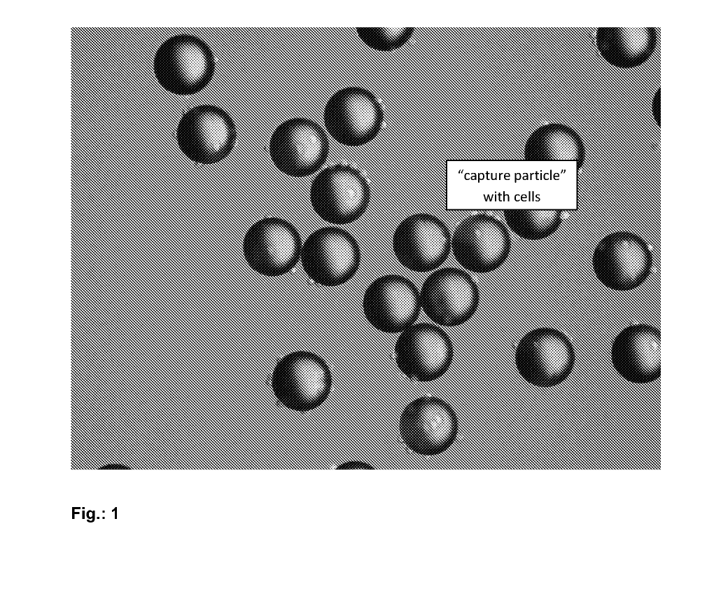

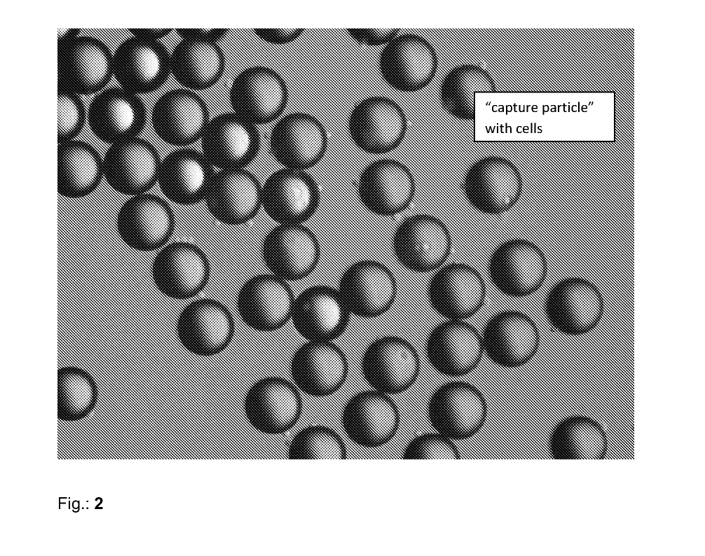

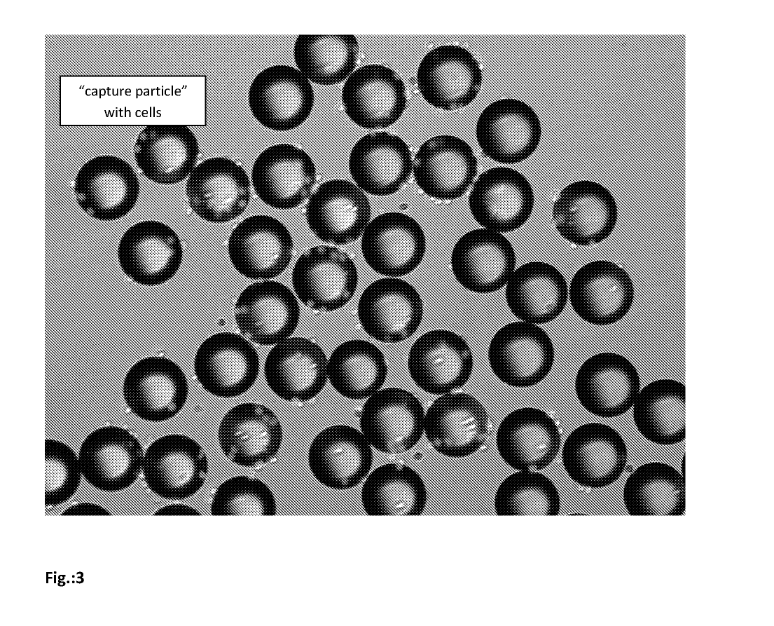

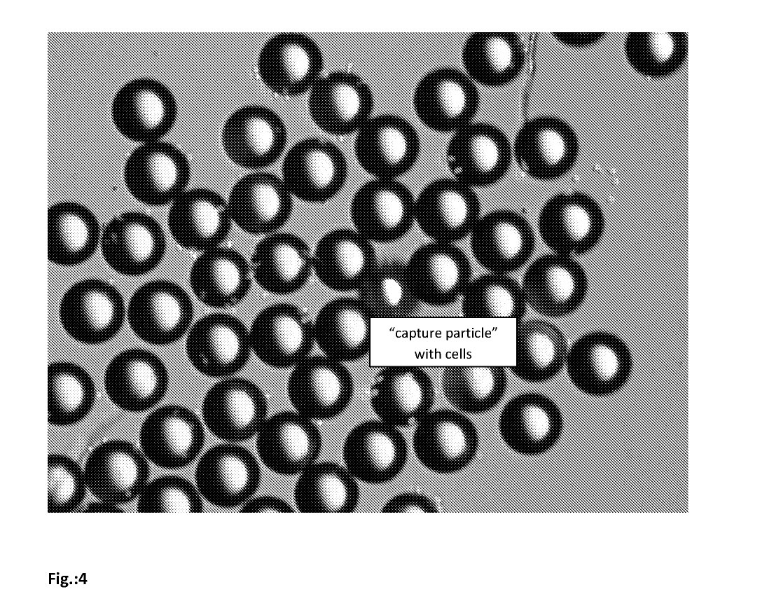

As can be seen in the FIGS. 1-4, it is possible to isolate MNC from the same identical by means of the total CIC protein fraction as well as the 30-100 kDa fraction. At least partially, these specific cells are not bound by antibodies but by proteins with a relative mole mass between 30 and 100 KDa.

All characteristics of the foregoing description and the following claims can be relevant both singular and in free combination for the realization of the invention for different embodiments.

1. A method to isolate at least one of somatic cells, microbes, and virus from whole blood comprising:
(a) isolating immune complexes comprising sub units from body fluid of an individual;
(b) cleaving the isolated immune complexes in their sub units;
(c) Fractionizing the sub units according to molecular weight and binding of a fraction of the dissociated sub units on a solid support;
(d) incubating the modified support from (c) with the blood of the individual from (a);
(e) isolating by a sieve the incubated support from (d) with at least one of cells, microbes, and/or virus bound to the sub units.

2. The method of the claim 1, further comprising step (f), selected from the group consisting of characterizing, cultivating in vitro, splitting in subpopulations, manipulating, and using for further diagnostic and therapeutic purposes the cells from step (e).

3. The method of claim 1, further comprising cleaving said immune complexes and binding them with known methods in dissociated form on a solid support.

4. The method of claim 1, wherein the body fluid is blood.

5. The method of claim 1, wherein the immune complexes are cleaved in the sub units by lowering the pH to a pH<3.0.

6. The method of claim 1, wherein the sub units without further processing or after fractionating according to molecular weight or affinity, are adsorptively or covalently bound on solid supports

7. The method of claim 1, wherein the solid support is selected from the group consisting of polystyrene, polyvinyl acrylate, polymethyl methacrylate, polylactide, and Sepharoses.

8. The method of claim 1, wherein all materials are suitable which are predetermined by the purpose for a diagnostic or therapeutic application.

9. The method of claim 1, wherein separation of the solid support and isolation of at least one of the cells, micro organisms and virus is accomplished by at least one member of the group consisting of sedimentation, magnetic enrichment, and separation by size differences, and the release of the cells is accomplished by lowering the pH or enzyme reactions.

10. The method of claim 1, further comprising:
obtaining circulating immune complexes from the plasma of an individual, by a procedure selected from the group consisting of precipitation procedures and protein A adsorbers;
after isolation, cleaving the CIC into their biologic active sub units, by lowering of the pH at ≦3.0, and separating them into their sub units by gel chromatography; and
coupling the subunits individually or as mixture on a microparticle solid support.

11. The method of claim 1, further comprising
attaining anti-coagulated blood by common blood drawing systems, centrifuging the whole blood, collecting the plasma supernatant, carrying out a PEG precipitation, centrifuging the precipitated fraction, and resolving the CIC containing sediment as mixture in a liquid;
lowering the pH of the CIC mixture from step (a) to 3.0; and
incubating NHS and EDC activated polystyrene particles with the dissociated sub units from step (b).

12. The method of claim 10, further comprising wherein, in step (b) after the dissociation of the CIC in their sub units, attaining a 30 kDa-100 kDa fraction by centrifugation, and using that fraction in the subsequent steps.

13. The method of claim 10, further comprising the steps of
(d) incubating the modified particles from step (c) with Mono Nuclear Cells (MNC) of the individual from step (a); and
(e) isolating the particles from step (d) with the bound MNC using a sieve.

14. The method of claim 10, further comprising the steps of
(d) incubating the modified particles from step (c) with whole blood of the individual from step (a), wherein said whole blood is concentrated by centrifugation and discarding of the plasma; and
(e) isolating particles from step (d) with the at least one bound cells, micro organisms, and virus by a sieve.

15. A device for isolation of at least one of somatic cells, microbes, and virus from whole blood comprising
a solid microparticle support;
sub units from immune complexes bound to the solid support, wherein said subunits are CIC from an individual; and
at least one of cells, micro organisms and virus, from the sa individual

16. Patient-specific diagnosis diagnostic and therapy, preferably for the detection and treatment of pathogen situations of the individual from whom the immune complexes are obtained, comprising performing on an individual in need of diagnosis and therapy the method of claim 1.

17. (canceled)

18. Methods, which are suitable to use in extra-corporeal circuit for the preparation or depletion of at least one member of the group consisting of somatic cells, micro organisms and virus which are identified by immune complex components, said methods comprising the method of claim 1.

19. The method of claim 1, further comprising analyzing said sub units for treatment of diseases, which are caused or maintained by the dysregulation of the immune system, including certain chronic virus diseases, preferably virus hepatitis, especially caused by Hepatitis C virus.

20. The method of claim 19, further comprising the step of providing extra-corporeal removal of at least one of immune complexes and their sub units and pro-inflammatory mediators.

21. (canceled)

22. A separation system for the isolation of at least one of somatic cells, micro organisms and virus from whole blood, wherein said system performs the steps (a)-(c) of claim 1 and, optionally, the following additional steps:
(d) incubating the modified particles from step (c) with whole blood of the individual from step (a), wherein said whole blood is concentrated by centrifugation and discarding of the plasma; and
(e) isolating particles from step (d) with the at least one bound cells, micro organisms, and virus by a sieve.

23. (canceled)
